(19) United States
(12) Patent Application Publication
(10) Pub. No.: US 2005/0019954 A1
(43) Pub. Date: Jan. 27, 2005

(54) Photochromic dyes for microsphere based sensor

(75) Inventors: Deepak Shukla, Webster, NY (US); Krishnan Chari, Fairport, NY (US); Samuel Chen, Penfield, NY (US)

Correspondence Address: Paul A. Leipold;Patent Legal Staff, Eastman Kodak Company, 343 State Street, Rochester, NY, 14650-2201, US

(73) Assignee: Eastman Kodak Company

(21) Appl. No.: 10/625,375

(22) Filed: Jul. 23, 2003

(51) Int. Cl. G01N 33/543

(52) U.S. Cl. 436/518

(57) ABSTRACT

A color coded bead for use in a microarray for detecting target analytes, the bead comprising: a photochromic compound in a medium, the bead having a receptor molecule on its surface; wherein the photochromic compound confers on the bead a distinct optical signature; and wherein the receptor molecule is capable of binding to a target analyte.

### CROSS REFERENCE TO RELATED APPLICATIONS

This application relates to commonly assigned copending application Ser. No. ______ (85504), entitled COLORABLE MICROSPHERES FOR DNA AND PROTEIN MICROARRAY, Ser. No. ______ (85677), entitled COLORABLE POLYMERIC PARTICLES WITH BIOLOGICAL PROBES and Ser. No. ______ (85486LMB), entitled POLYMER MICROSPHERES CONTAINING LATENT COLORANTS AND METHOD OF PREPARATION filed simultaneously herewith. The copending applications are incorporated by reference herein for all that they contain.

### FIELD OF THE INVENTION

The present invention in general is concerned with microsphere based sensor technology for the detection of gaseous or liquid analytes. In particular, the invention concerns a sensor array of microspheres containing photochromic materials.

### BACKGROUND OF THE INVENTION

Array based sensors provide an architecture for multi-analyte sensing (Chem. Rev. 100, 2695-2626, 2000). The ability to more efficiently detect and analyze specific components (analytes) of a mixture or sample would greatly benefit medicine, environmental analysis, and consumer industries (e.g., food analysis), to name a few. For example, the food industry depends upon chemical analysis for quality control, environmentalists depend upon chemical analysis for the detection of harmful agents in natural resources, such as water, and the medical community depends upon analysis for the detection of agents such as metabolites, drugs, and glucose to name a few. The basic principles of protein micro array assays were already described at the end of the eighties by Roger Ekins (J Pharm Biomed Anal 7, 155-168, 1989) for immunological microarray assays. The interest in microarray-based assays increased enormously with the development of DNA chip technology.

Since it was invented in the early 1990s (Science, 251, 767, 1991), high density arrays formed by spatially addressable deposition of sensors on a two-dimensional solid support has greatly enhanced and simplified the process of array based sensor technologies. The key to current microarray technologies is the placement of receptors at predetermined location. The presence or absence of an analyte is then discerned by monitoring a specific location of a sensor array of receptors. All of these systems require preparing a sensor array with a plurality of receptors at predetermined locations and involve complex and expensive processing steps.

For example, Park, et al. (Science, 276,1401, 1997) has disclosed a lithographic method for producing arrays of nanometer-sized holes using polystyrene-polybutadiene, ordered, diblock copolymers as masks in reactive ion etching of silicon nitride. This multi-step method is capable of producing arrays of picoliter-sized holes which are typically 20 nanometers in diameter and 20 nanometers deep with spacing of 40 nanometers. Hole densities of up to 10″ holes/cm² are disclosed. The range of sizes and spacings of the holes produced by this method is limited by the size of the copolymer microdomains. Uniformity of hole size and spacing is difficult to maintain with this method due to difficulties in controlling the etching method employed to form the holes.

Deutsch, et al. (Cytometry, 16, 214, 1994) have disclosed a porous electroplated nickel microarray comprised of micron-sized conical holes in blackened nickel plate. Hole sizes range from a 7 μm upper diameter to a 3 μm lower diameter with an 8 μm depth. The array is used as a cell carrier for trapping individual cells while studying the responses of individual cells to changes in their microenvironment. U.S. Pat. No. 4,772,540 to Deutsch et al., also discloses a method for making such an array using a combined photoresist and electroplating technique.

Corning Costar Corp. (Acton, Mass.) produces a commercial microwell array for miniaturized assays under the trademark PixWell®. These arrays are made from microformed glass plates and comprise 40 μm diameter by 20 μm deep tapered wells with a well density of 4356 wells/cm².

Microwell arrays have particular utility in the study of living cells. In cell research, the measurement of responses of individual cells to changes or manipulations in their local environment is desirable. Any method or device designed for such studies must provide for the capability of maintaining cell viability, identifying the location of individual cells, and correlating response measurements with individual cells.

An alternative approach to the spatially addressable microwell arrays method is the concept of using fluorescent dye-incorporated polymeric beads to produce biological multiplexed arrays. U.S. Pat. No 5,981,180 discloses a method of using color coded beads in conjunction with flow cytometry to perform multiplexed biological assay. Microspheres conjugated with DNA or monoclonal antibody probes on their surfaces were dyed internally with various ratios of two distinct fluorescence dyes. Hundreds of “spectrally addressed” microspheres were allowed to react with a biological sample and the “liquid array” was analyzed by passing a single microsphere through a flow cytometry cell to decode sample information. U.S. Pat. No. 6,023,540 discloses the use of fiber-optic bundles with pre-etched microwells at distal ends to assemble dye loaded microspheres. The surface of each spectrally addressed microsphere was attached with a unique bioactive agent and thousands of microspheres carrying different bioactive probes combined to form “beads array” on pre-etched microwells of fiber optical bundles. More recently, a novel optically encoded microsphere approach was accomplished by using different sized zinc sulfide-capped cadmium selenide nanocrystals incorporated into microspheres (Nature Biotech. 19, 631, 2001). Given the narrow band width demonstrated by these nanocrystals, this approach significantly expands the spectral barcoding capacity in microspheres.

Even though the color coded or “spectrally addressed” microsphere approach provides an advantage in terms of its simplicity over microwell “spatially addressable” approach in microarray making, there are still needs in the art to make the manufacture of sensor microarrays less difficult and less expensive.

U.S. Ser. No. 09/942,241 provides a microarray that is less costly and easier to prepare than those previously disclosed because the support need not be modified; nevertheless the microspheres remain immobilized on the substrate. U.S. Ser. No. 09/942,241 provides a microarray comprising: a substrate coated with a composition comprising microspheres dispersed in a fluid containing a gelling agent or a precursor to a gelling agent, wherein the microspheres are immobilized at random positions on the substrate. The substrate is free of receptors designed to physically or chemically interact with the microspheres. The invention discloses a unique coating composition and technology to prepare a microarray on a substrate and does not require placement of microspheres at predetermined locations as.

U.S. Ser. No. 09/942,241 teaches various coating methods but exemplifies machine coating, whereby a support is coated with a fluid coating composition comprising microspheres dispersed in gelatin. Immediately after coating, the support is passed through a chill set chamber in the coating machine where the gelatin undergoes rapid gelation and the microspheres are immobilized.

Although this invention provides an immense manufacturing advantage over other existing technologies, there are some limitations as well. Thus, like other current approaches of making microspheres based microarray, it involves color coding of individual microsphere, and the color intensity and hue are associated with a unique biological probe covalently attached to the surface of the microsphere. However, such approach suffers from two major drawbacks. First, the dye used to color code microsphere itself emits fluorescence that interferes with the fluorescence signal resulted from the biological interaction; and secondly, the intensity of fluorescence emission signals resulting from the biological interaction is severely suppressed when the color coding dye in the microsphere is lower in energy. These problems severely limit the color coding diversity of the microspheres, and dramatically reduce the dynamic range and lower bounds of detectability of analyte in the microarray system. Even if it is possible to find non-fluorescent dyes for color coding microspheres, the problem of quenching of analyte-generated fluorescence emission by lower energy (or longer wavelength absorbing) dyes still remains.

In general, in any color-coded microsphere based sensor array where analyte-sensor interaction is measured by the resulting fluorescence emission, it is imperative that there is no undesired fluorescence from the dyes used to color-code the microspheres. However, it is difficult to find a set or class of dyes that will not fluoresce to some extent once incorporated in a rigid media like microspheres. In fact, even dyes with fluorescence lifetimes as short as a few picoseconds in solution, once incorporated into microspheres exhibit significant undesirable fluorescence.

There is a need for alternative ways to color code microspheres used in microarrays. It is accordingly, an object of this invention to overcome the problems discussed above by providing a novel methodology to color code microspheres using photochromic compounds.

Another object of this invention is to provide methodology to color code microspheres using mixtures of photochromic compounds and mixtures of photochromic and non-photochromic dyes that do not produce interfering fluorescence.

“Photochromism is a reversible transformation of a chemical species induced in one or both directions by absorption of electromagnetic radiation between two forms, A and B, having differenta absorption spectra” (Pure Appl. Chem., 73, 639, 2001). This reaction gives rise to the formation of photoisomers whose electronic absorption spectra are markedly different from that of the reactant molecule, which results in the dramatic color change. As for organic photochromic molecules, the reactant is generally colorless, meaning that its electronic absorption starts only from the UV region (<350 nm), while the products induced by the UV irradiation show the intense absorption in the visible region (400 to 700 nm). Changes in other molecular properties such as reflective and dielectric constants can also occur along with the color change during the photochromic reaction. Reversibility is the main criterion for photochromism. While the forward reaction is always photochemical, the back reaction is induced mostly by a thermal mechanism at room temperature. Some photochromic molecules yield thermally stable photoproducts, in such cases back reactions are photochemical. These phenomena have received great attention because photochromic compounds have a wide variety of commercial applications: ophthalmic glasses, optical switches, optical memories, and nonlinear optical devices.

### SUMMARY OF THE INVENTION

According to the present invention, there is provided a method for color coding microspheres using photochromic materials. On the surface of microspheres are receptor molecules that are capable of binding to an analyte of interest and to create a signal.

A microsphere-based sensor array system is disclosed in which microspheres carry different receptor molecules (probes) that retain their unique identity even after they are mixed together. The analyst is able to identify the functionality (probe) on each microsphere by using photochromic compounds as an optically interrogatable encoding scheme.

The invention discloses a color coded bead for use in a microarray for detecting target analytes, the bead comprising:
a photochromic compound in a medium; the bead having a receptor molecule on its surface;
wherein the photochromic compound confers on the bead a distinct optical signature; and
wherein the receptor molecule is capable of binding to a target analyte.

Another aspect of the invention discloses a microarray for detecting analytes, the microarray comprising a support, on which are disposed the beads defined in the above embodiment of the invention.

Yet another aspect of the invention discloses a method of identifying target analytes, the method comprising the steps of:

a) providing the microarray defined above, wherein the beads carry receptor molecules capable of binding to the target analytes;

b) enabling the target analytes to bind to the receptor molecules thereby generating optical signals;

c) recording the signals as Image A;

d) activating the photochromic compounds in the beads into color signatures and recording them as Image B; and

e) matching Images A and B to determine the identity of the analyte.

In general, in any color-coded microsphere based sensor array where analyte-sensor interaction is measured by the resulting fluorescence emission, it is imperative that there is no undesired fluorescence from the dyes used to color-code the microspheres. However, it is difficult to find a set or class of dyes that will not fluoresce to some extent once incorporated in a rigid media like microspheres. In fact, even dyes with fluorescence lifetimes as short as a few picoseconds in solution, once incorporated into microspheres, exhibit significant undesirable fluorescence.

Photochromic compounds are generally colorless, meaning that its electronic absorption starts only from the UV region (<350 nm), while the colored photoisomers generated by the UV irradiation show intense absorption in the visible region (400 to 700 nm). By using photochromic compounds in micropheres, actinic radiation can be used to switch on, or develop, the color that is used to code the microspheres. Color can be switched on only when color is required (or on demand). This method for color-coding microspheres affords tremendous advantage over previous technologies.

One advantage is that since the color in microsphere can be generated on demand by using actinic radiation, the present invention allows for interference- or noise-free monitoring (or detection) of fluorescence signal generated from sensor-analyte interaction. Another advantage is that the fluorescence signal intensity generated from the analyte-sensor interaction is not attenuated or quenched. Yet another advantage is that the color in the microspheres can be bleached thermally or photochemically. Importantly, since the process with photochromic dyes is completely reversible, analyte-sensor emission detection and color-code generation in microspheres can be repeated.

### BRIEF DESCRIPTION OF THE DRAWINGS

FIG. 1 is a schematic showing the steps of the invention.

FIG. 2 is an electromagnetic spectral response of beads containing a mixture of photochromic and non-photochromic compounds before (curve A) and after (curve B) actinic radiation.

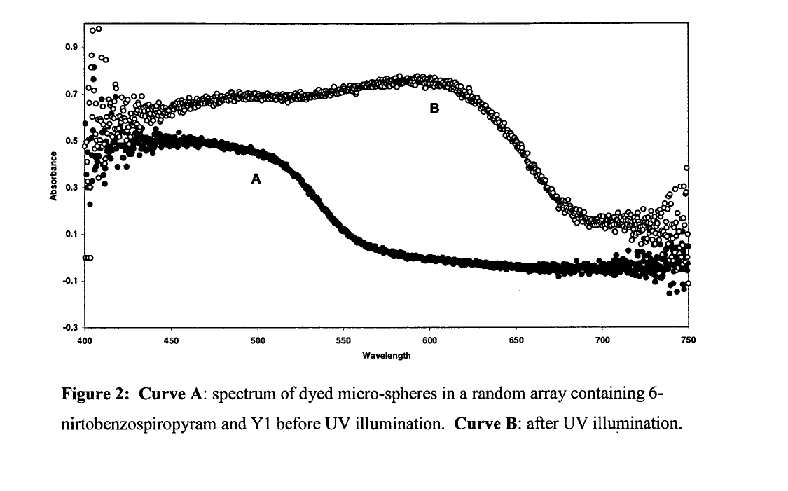

### DETAILED DESCRIPTION OF THE INVENTION

The present invention improves on two related technologies in the art: 1) the development of a polymeric bead- or microsphere-based sensor array in which crospheres, also termed beads, carrying different receptor molecules may be mixed together and still retains their identity, so that an analyst may determine the identity of the receptor molecules on each microsphere by using optically interrogatable encoding scheme; and 2) the development of a microarray comprising a support having a surface on which the polymeric beads are immobilized in a random or ordered pattern.

In general, according to one embodiment, the invention concerns a sensor array system that comprises a population of microspheres. On the surface of the microspheres are receptor molecules which may bind to targeted analytes. Binding may occur by a variety of chemical or physical interactions such as, hydrogen bonding, electrostatic interactions or covalent bonding. Binding is detected by an optical signal.

Different mechanisms can generate the optical signal. Examples include the binding of a dye-tagged analyte to the receptor molecule on a microsphere; the destruction of existing dye species on a microsphere; and a change in optical signal upon analyte interaction with receptor molecule on the microsphere. Although the microspheres may be randomly mixed together, the receptor molecule on each is determined via a photochromic color-code which is encoded with a description of the receptor molecule. As a result, by observing whether the optical signature of a particular microsphere is exhibiting a change, or not, and then decoding the photochromic color-code signature for the receptor molecule on the microsphere, the presence or absence of analyte targeted by the functionality may be determined.

In one embodiment of the invention, the microspheres are encoded using photochromic compounds or a mixture of photochromic and non-photochromic compounds, that are incorporated in the microsphere. On the surface of the microspheres are receptor molecules.

In another embodiment, the present invention discloses a microsphere based random microarray on a support with each polymeric bead in the microarray having an optical signature that identifies a probe on its surface. The optical signature is generated using a photochromic compound by itself, or by using a mixture of photochromic compounds, or a mixture of photochromic and non-photochromic compounds.

In the context of this invention, the term “microarray” or “array” means a collection of random or ordered microspheres that are composed of an organic or inorganic material on a surface of a 2-dimensional support. The preferred material for the microspheres is a polymer matrix. The microspheres are incorporated with one, or more than one, photochromic compounds or a mixture of photochromic and non-photochromic compounds. On the surface of the microspheres are receptor molecules that are capable of binding to the analyte of interest (target analyte), and by binding, create an optical signal. An array typically contains microspheres of more than one type with more than one type of receptor molecule. The size and shape of an array can vary depending on the composition and intended use. In addition, an array may contain multiple sub-arrays in various formats.

In the present invention, the distribution or pattern of the polymeric beads on the support can be ordered or entirely random. The polymeric beads are immobilized in a 2-dimesional plane on the surface of a support. The possible supports include, but are not limited to, glass, metals, polymers, and semiconductors. The support can be transparent or opaque, flexible or rigid. In some cases, the support can be a porous membrane e.g. nitrocellulose and polyvinylidene difluoride. The polymeric beads are immobilized on the surface of the support by physical or chemical interactions between the support and the polymeric beads. To improve robustness and reproducibility, it is more desirable to immobilize the polymeric beads onto a modified surface using certain chemical functional agents, that is, the surface is chemically treated or modified to allow attachment of the polymeric beads. As will be appreciated by those skillful in the art, the surface can also be modified to provide physical forces, e.g. electrostatic, magnetic, compressive, adhesive, etc., that allow the attachment of the polymeric beads to such modified surfaces. Generally the support surface is planar, however it can also be a modified surface that contains regular or irregular 3-dimensional configurations, for example micro wells or cavities can be used to immobilize the polymeric beads on a surface by embedding the polymeric beads into the wells. The polymeric bead can also be immobilized on a 2-dimensional plane by allowing the polymeric bead to flow through a confined space, e.g. a tube or a chamber, that allows the polymeric beads to assemble into a 2-dimensional array.

In a preferred embodiment, the polymeric beads are immobilized on the surface using a coating method through a “sol-to-gel” transition process. As used herein, the term “sol-to-gel transition” or “gelation” means a process by which fluid solutions or suspensions of particles form continuous three-dimensional networks that exhibit no steady state flow. This can occur in polymers by polymerization in the presence of polyfunctional monomers, by covalent cross-linking of a dissolved polymer that possesses reactive side chains and by secondary bonding, for example, hydrogen bonding, between polymer molecules in solution. Polymers such as gelatin exhibit thermal gelation that is of the latter type. The process of gelation or setting is characterized by a discontinuous rise in viscosity. (See, P. I. Rose, “The Theory of the Photographic Process”, 4^{th} Edition, T. H. James ed. pages 51 to 67).

As used herein, the term “gelling agent” means a substance that can undergo gelation as described above. Examples include materials such as gelatin, water-soluble cellulose ethers or poly(n-isopropylacrylamide) that undergo thermal gelation, or substances such as poly(vinyl alcohol) that may be chemically cross-linked by a borate compound. Other gelling agents may be polymers that may be cross-linked by radiation such as ultraviolet radiation. Examples of gelling agents include acacia, alginic acid, bentonite,carbomer, carboxymethylcellulose sodium, cetostearyl alcohol, colloidal silicon dioxide, ethylcellulose, gelatin, guar gum, hydroxyethylcellulose, hydroxypropyl cellulose, hydroxypropyl methylcellulose, magnesium aluminum silicate, maltodextrin, methylcellulose, polyvinyl alcohol, povidone, propylene glycol alginate, sodium alginate, sodium starch glycolate, starch, tragacanth and xanthum gum. (For further discussion on gelling agents, see, accompanying reference Secundum Artem, Vol. 4, No. 5, Lloyd V. Allen). A preferred gelling agent is alkali pretreated gelatin.

The microspheres or beads may comprise any suitable material organic or inorganic. Preferably the microspheres are made from polymeric materials. Suitable methods for preparing the polymeric microspheres are emulsion polymerization as described in “Emulsion Polymerization” by I. Piirma, Academic Press, New York (1982) or by limited coalescence as described by T. H. Whitesides and D. S. Ross in J. Colloid Interface Science, vol. 169, pages 48-59, (1985). The particular polymer employed to make the particles or microspheres is a water immiscible synthetic polymer that may be colored. The preferred polymer is any amorphous water immiscible polymer. Examples of polymer types that are useful are polystyrene, poly(methyl methacrylate) or poly(butyl acrylate). Copolymers such as a copolymer of styrene and butyl acrylate may also be used. Polystyrene polymers are conveniently used.

Although microspheres or polymeric beads having a substantially curvilinear shape are preferred because of ease of preparation, particles of other shape such as ellipsoidal or cubic particles may also be employed.

The microspheres are desirably formed to have a mean diameter in the range of 1 to 50 microns; more preferably in the range of 3 to 30 microns and most preferably in the range of 5 to 20 microns. It is preferred that the concentration of microspheres in the coating is in the range of 100 to a million per cm², more preferably 1000 to 200,000 per cm² and most preferably 10,000 to 100,000 per cm².

The microspheres may be purchased with the desired functionality (receptor molecule) already present. Alternatively, “blank” microspheres may be used that have surface chemistries that facilitate the attachment of desired receptor molecules (probes) by the user.

It should be understood that receptor molecules may be present throughout the microsphere's volume, and not limited to the physical circumferential surface.

In the prior art, a large spectrum of receptor molecules have been manifest on microspheres that produce optically interrogatable changes in the presence of the targeted analyte. These receptor molecules include four broad classifications of microsphere sensors: 1) basic indicator chemistry sensors; 2) enzyme-based sensors; 3) immuno-based sensors; and 3) geno-sensors.

A large number of basic indicator microsphere sensors have been previously demonstrated. Examples include:

TABLE 1
Chemical Sensors

| | Examples of Suitable Receptor |
|---|---|
| Type of Sensor | Molecules |
| pH Sensors: | seminaphthofluorescein |
| | fluorescien |
| | 8-hydroxy-pyrene-1,3,6-trisulfonic acid |
| CO₂ Sensors | seminaphthofluorescein |
| | 8-hydroxy-pyrene-1,3,6-trisulfonic acid |
| Metal ion sensors | cyclen derivatives |
| | derivetized peptided |
| | calcine blue |
| | methyl calcine blue |
| | bis(salicylidine ethylenediamine) (SED) |
| | Indo-1 |
| | Flura-2 |
| | Magnesium green |
| O₂ Sensors | Phenylisobenzofuran |
| | Methoxyvinyl pyrene |
| Nitric Oxide (NO) sensors | luminol |
| | dihydrorhodamine |
| Ca²⁺ sensor | Bis-Fura |
| | Indo-1 |
| | Fluo-3 |
| | Rhod-2 |
| Zn²⁺ sensor | Newport Green |
| Na⁺ sensor | SBFI |
| | SBFO |
| | sodium green |

Each of the chemicals listed in Table 1 directly produces an optically interrogatable signal or signal change in the presence of targeted analyte.

Enzyme based microsphere sensors have also been demonstrated and could manifest on microspheres. As shown in Table 2, examples include:

TABLE 2
Enzyme Sensors

| | Examples of Suitable |
|---|---|
| Type of Sensor | Receptor Molecules |
| Glucose | Glucose oxidase (enz.) + O₂ sensitive |
| | dye (see Table 1) |
| Pencillin | Pencillinase (enz.) + pH sensitive dye |
| | (see Table 1) |
| Urea | Urease (enz.) + pH sensitive dye (see |
| | Table 1) |
| Acetylcholine | Acetylcholinesterase (enz) + pH |
| | sensitive dye (see Table 1) |

Generally, the induced change in the optical signal due to the binding of enzyme sensitive chemical analytes occurs indirectly in this class of receptor molecules. For example, the microsphere bound glucose oxidase, decomposes the target analyte. e.g., glucose, consume a co-substrate, e.g., oxygen, or produce some by product, e.g., hydrogen peroxide. An oxygen sensitive dye is then used to trigger the signal change. Techniques for immobilizing enzymes on microspheres, are known in art.

Immuno-based microsphere sensors have been demonstrated for the detection of environmental pollutants such as pesticides, herbicides, PCB's and PAH's. Additionally, these sensors have also been used for diagnostics, such as bacterial (e.g., cholera, lyme disease, tuburculosis), viruses (e.g., HIV, herpes simplex, cytomegalovirus), fungal (e.g., mycoplasmal, pneumonias), protozoal (e.g., amoebiasis, taxoplasmosis), rickettsial, and pregnancy tests.

Microsphere based genosenory array have also been demonstrated. These are typically constructed by attaching a probe sequence to the microsphere surface (typically via NH₂ group). A fluorescent dye molecule, e.g., fluorescein, is attached to the target sequence, that is in solution. The optically interrogatable signal change occurs with the binding of target sequence to the microsphere. A few demonstrated probe and target sequences (see Ferguson, J. A. et al. Nature Biotech. 14, 1996). Alternatively, upon binding of the target sequences, an intercalating dye (for example, ethidium bromide) can be added subsequently to signal the presence of bound target to the probe sequence.

The microspheres exhibiting activity or change in their optical signal may be identified by decoding their color-code by exposure of the photochromic composition to actinic radiation.

The random or ordered array of microspheres, each carrying a different receptor molecule (probe), is brought into contact with an analyte of interest to which some of the receptors molecules may interact. Any binding changes the optical signal of the corresponding microsphere. The microspheres exhibiting activity or change in their optical signal may be identified by decoding their color-code by exposure of photochromic composition to actinic radiation. By identifying the receptor molecule of the microsphere in which the optical signal has changed, using the encoded photochromic compounds composition, the information regarding the chemical identity and concentration of the analyte may be gained based upon the binding or non-binding to the receptor molecule.

Thus, according to the present invention, a microsphere further comprises one or more than one photochromic compound in the microsphere as optical signatures. Furthermore, a microsphere may comprise one or more than one photochromic compound along with one or more non-photochromic compounds in the microsphere as optical signal. As used herein, the term “photochromic compound” means a molecule with absorption and emission characteristics that can be modulated using actinic radiation. It is preferred that the photochromic compound be colorless and not fluoresce. As used herein, the term “optical signal” means an absorption or emission signal that can be measured through optical methods. Such signals include, but are not limited to, absorbence, fluorescence, change in refractive index and chemiluminescence. Both the ratio and the concentration of the mixture of the photochromic compounds can be varied to generate a library of unique optical signature encoded polymeric beads. As such, each microsphere in the library is associated with a unique sensor probe. A photochromic compound can be organic or inorganic, and may be polymeric. A photochromic compound is associated with a microsphere by either covalent binding or non-covalent interaction, either on the surface of the microsphere or incorporated inside the microsphere. In a preferred embodiment, a photochromic compound is incorporated into a microsphere using a loading process.

Actinic radiation is used to switch photochromic compounds into colored compounds (forms) to generate detectable optical signatures. Examples include, but are not necessarily limited to, photochromic dye formation as reviewed in Photochromism: Molecules and Systems, Durr and Bouas-Laurent edited, Elsevier, Amsterdam, (1990).; by Durr and Bouas-Laurent in a IUPAC Technical Report in Pure Appl. Chem., 73, 639, 2001.

In an alternative embodiment, additional encoding parameters can be added, such as microsphere size. If a number of sensors needed exceeds a few hundred, it is possible to use microspheres of different sizes to expand the encoding dimensions of the microspheres. In this manner, the same photochromic composition could be used to encode microspheres of different sizes thereby expanding the number of different receptor molecules/sensors present in the array.

The photochromic materials which can be used in the present invention include organic photochromic substances such as dihydropyrene compounds, 1,4-2H-oxazine, spirothiopyran compounds, naphthopyran compounds, triphenylmethane compounds, benzopyran compounds, azobenzene compounds, dithizone metal complex compounds, thioindigo compounds, spirooxazine compounds, spiropyran compounds, diarylethylene compounds and fulgide compounds. Some specific examples of photochromic compounds are discussed below.

The diarylethylnes are useful photochromic compounds to color code microspheres in the present invention. These fulgides have the general formula: 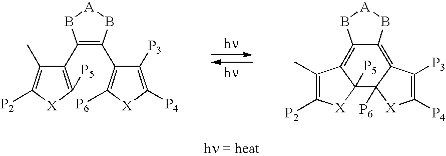
wherein A represents alkylene group having 1 to 3 carbon atoms or alkylene groups having 1 to 3 carbon atoms substituted with fluorines, oxygen or NR₁, where R₁ represents hydrogen, alkyl having 1 to 20 carbon atoms, cycloalkyl having 5 to 12 carbon atoms, arylalkyl having 7 to 9 carbon atoms, aryly having 6 to 14 atoms which may be substituted with one or more hydrogen or alkoxy groups having 1 to 20 carbon atoms. B represents CH₂, CF₂, CN or CO. X represents sulfur, oxygen, and NR₂, where R₂ represents hydrogen, alkyl having 1 to 20 carbon atoms, cycloalkyl having 5 to 12 carbon atoms, arylalkyl having 7 to 9 carbon atoms, aryly having 6 to 14 atoms which may be substituted with one or more hydrogen or alkoxy groups having 1 to 20 carbon atoms and P₁, P₂, P₃, P₄, P₅ and P₆ independently represent hydrogen, alkyl, aryl groups etc. These compounds are well known in the art as are methods for their preparation (for example, Japan Kokai Tokkyo Koho JP 9761647 A2, JP 9780681 A2)

The spiropyrans compounds are particularly useful as photochromic dyes in the present invention. These compounds have the general formulae: 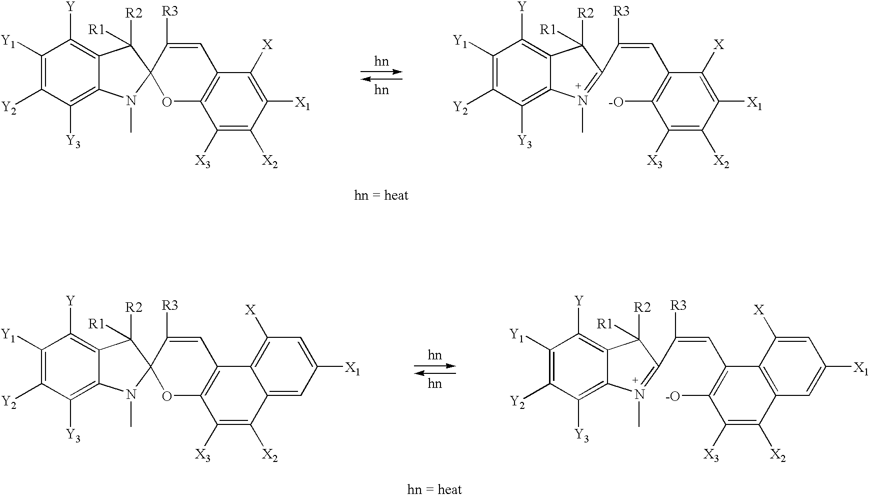
wherein, R, R1 and R2 represent aryl groups, the same or different alkylgroups having 1 to 20 carbon atoms, and R1 and R2 taken together could forma a saturated carbocyclic ring, R3 is hydrogen or an alkyl group having 1 to 20 carbon atoms, inclusive, X, X1, X2, X3, Y, Y1, Y2, and Y3 represent hydrogen, aryl, cyano, or carboxyl groups, an alkoxyl group having 1 to 20 carbon atoms, a nitro group or a halogen group. These compounds are well known in the art as are methods for their preparation. For example U.S. Pat. Nos. 2,953,454 and 3,022,318 various compounds and methods for their preparation. Examples of some benzospiropyrans useful for color coding microspheres are, 6-nitro-8-methoxy-1′,3′,3′-trimethylspiro[2H-1-benzopyran-2,2′-indoline], 6-nitro-8-methoxy-5′-chloro-1′,3′,3′-trimethylspiro[2H-1-benzopyran-2,2′-indoline], 6-nitro-8-methoxy-5′-bromo-1′,3′,3′-trimethylspiro[2H-1-benzopyran-2,2′-indoline], 6-nitro-8-methoxy-5-bromo-5′-chloro-1′,3′,3′-trimethylspiro[2H-1-benzopyran-2,2′-indoline], 6,5′-nitro-8-methoxy-1′,3′,3′-trimethylspiro[2H-1-benzopyran-2,2′-indoline], 6-nitro-8-ethoxy-1′,3′,3′-trimethylspiro[2H-1-benzopyran-2,2′-indoline].

Other spiropyran in this class are derivatives of following classes of compounds spiro[2H-1-benzopyran-2,2′-[1H]-benzo[g]indoline], spiro[2H-benzopyran-2,2′-[1H]-benzo[e]indoline], spiro[indoline-2′,3′-[3H]-naphtho[2,1-b]pyran], spiro[2H-1-benzopyran-2,2′-benzothiazolines], spiro[benzothiazoline-2,3′-[3H]-naphthao[2,1-b]pyran, 2,2′-spirobi[2H-1-benzopyran], 3,3′-spirobi[3H-naphthao[2,1-b]pyran], 3,3′-spirobi[3H-naphthao[1,2-b]pyran], spiro[2H-1-benzopyran -2,3′-[3H]-naphtho[2,1-b]pyran], spiro[2H-1-benzopyran-2,3′-[3H]-naphtho[1,2-b]pyran], spiro[4H-1-benzopyran-4,3′-[3H]-naphtho[2,1-b]pyran], spiro[2H-naphtho[1,2-b]pyran-2,3′-3[H]-naphtho[2,1-b]pyran], spiro[indoline-2,2′-pyrano[3,2-H]quinoline], spiro[2H-1-benzopyran-2,2′-[2H]quinoline].

The spirooxazine class of photochromic dyes are also useful for color coding microspheres in the invention. These photochromic dyes have the general formula: 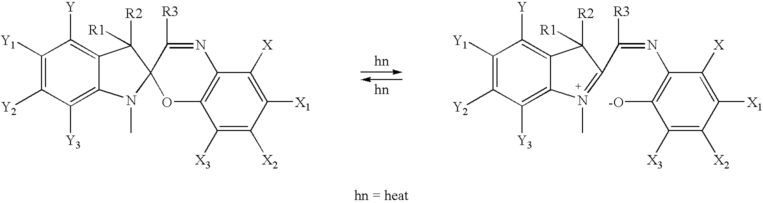
wherein, R, R1 and R2 represent aryl groups, the same or different alkylgroups having 1 to 20 carbon atoms, and R1 and R2 taken together could forma a saturated carbocyclic ring, R3 is hydrogen or an alkyl group having 1 to 20 carbon atoms, inclusive, X, X1, X2, X3, Y, Y1, Y2, and Y3 represent hydrogen, aryl, cyano, or carboxyl groups, an alkoxyl group having 1 to 20 carbon atoms, a nitro group or a halogen group. Methods for preparation of these compounds can be found for example in U.S. Pat. No. 3,562,172 and 3,578,602. Some useful spirooxazines are: 1,3,3,-trimethylspiro[indolino-2,3′-naphtho[2,1-b](1,4)-oxazine], 1,3,3,5-tetramethylspiro[indolino-2,3′-naphtho[2,1-b](1,4)-oxazine], 5-methoxy -1,3,3,-trimethylspiro[indolino-2,3′-naphtho[2,1-b](1,4)-oxazine], 1-β-carboxyethyl -3,3,-dimethylspiro[indolino-2,3′-naphtho[2,1-b](1,4)-oxazine], 1-β-carboxyethyl -3,3,5-trimethylspiro[indolino-2,3′-naphtho[2,1-b](1,4)-oxazine], 1-carboxyethyl -3,3,-dimethyl-5-methoxyspiro[indolino-2,3′-naphtho[2,1-b](1,4)-oxazine].

Spirothiopyran are also useful as photochromic dyes in present invention. These photochromic dyes have the general formula: 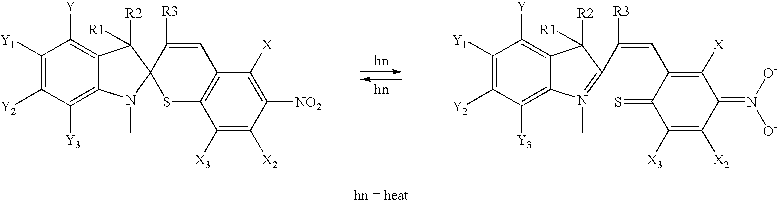
wherein, R, R1 and R2 represent aryl groups, the same or different alkylgroups having 1 to 20 carbon atoms, and R1 and R2 taken together could forma a saturated carbocyclic ring, R3 is hydrogen or an alkyl group having 1 to 20 carbon atoms, inclusive, X, X1, X2, X3, Y, Y1, Y2, and Y3 represent hydrogen, aryl, cyano, or carboxyl groups, an alkoxyl group having 1 to 20 carbon atoms, a nitro group or a halogen group. Methods for preparation of these compounds can be found for example in U.S. Pat. 4,565,779. Some useful spirooxazines are: 1′,3′,3′-trimethyl-6-nitrospiro-[2H-1-benzothiopyran-2,2′-indoline], 5′-methoxy-1′-n-hexyl-3′,3′-dimethyl-6-nitro-8-methoxyspiro-[2H-1-benzothiopyran-2,2′-indoline], 5′,7′-dimethoxy-1′,3′,3′-dimethyl-6-nitro-8-methoxyspiro-[2H-1-benzothiopyran -2,2′-indoline], 5′-methoxy-1′,3′,3′-dimethyl-6-nitro-8-chlorospiro -[2H-1-benzothiopyran-2,2′-indoline], 5′-dimethylamino-1′,3′,3′-dimethyl -6-nitrospiro-[2H-1-benzothiopyran-2,2′-indoline].

Once the photochromic compounds are incorporated into microspheres, the identity of each type of microsphere can be distinguished by actinic exposure of photochromic compounds to generate corresponding colored compounds. The colored forms generate a unique optical signature that is measurable through physical means. Since on the surface of microspheres are receptor molecules capable of binding to the analyte of interest, each microsphere with a specific composition of a photochromic compound or a mixture of photochromic compounds or a mixture of photochromic and non-photochromic compounds corresponds to a specific receptor molecule. These microspheres may be mixed in equal amounts and the microarray fabricated by immobilizing the mixed microspheres on a 2- or 3-dimensional surface in a single or multilayer format.

The photochromic materials can be used independently; however, for the purpose of increasing the degree of saturation of the color change or for increasing the photochromic lifetime, it is desirable to use additional compounds such as plasticizers, hindered amines, hindered phenols, excited state quenchers, and so on. These materials are well known light stabilizers of photochromic compounds and their concentration may be selected from the well documented ranges (see for example U.S. Pat. Appl. 2003/0030040).

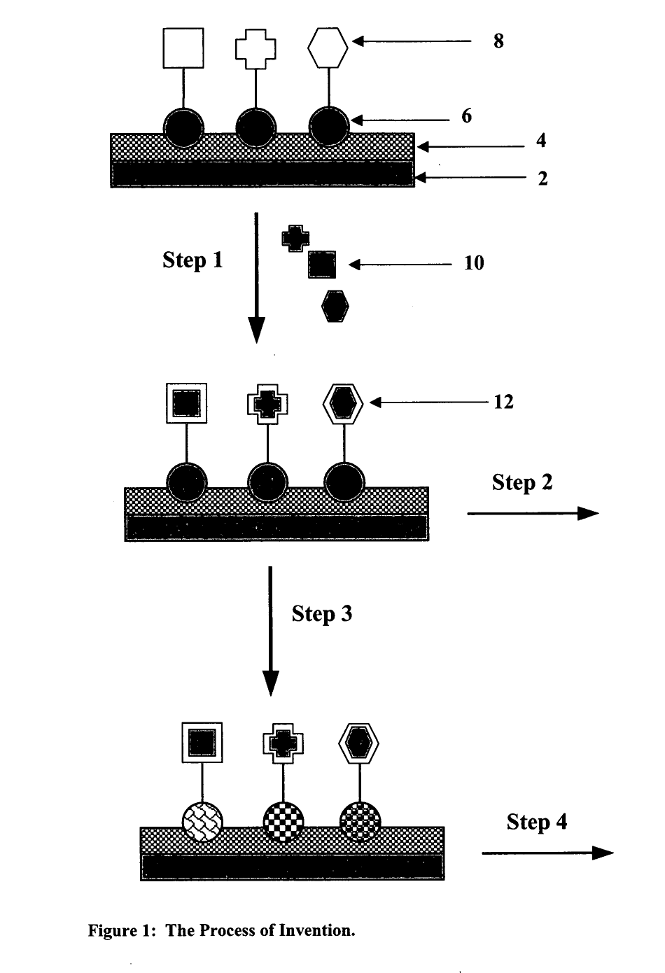

The invention further discloses a process of using such microarray. The random or ordered array of microspheres coded with photochromic composition 6 each carrying a different receptor molecule (probe) 8 is allowed to interact with analytes 10 of interest to which some of the receptors molecules may bind 12. Any interaction changes the optical signal of the corresponding microsphere. The microspheres exhibiting activity or change in their optical signal may be identified by decoding their photochromic color-code by exposure to actinic radiation. By identifying the receptor molecule of the microsphere in which the optical signal has changed, using the encoded photochromic composition, the information regarding the chemical identity and concentration of the analyte may be gained based upon the binding or non-binding to receptor molecule. The inventive process is schematically shown in FIG. 1.

In Step 1, the random array of microspheres coded with photochromic composition 6 each carrying a different receptor molecule 8 is allowed to interact with analyte 10 of interest to which some of the receptors molecules 8 may interact. This step requires good physical contact of the coated microarray with the analyte sample by either placing a layer of sample solution on the coated side of the microarray or dipping the microarray into the sample solution. In Step 2, the targeted analytes 10 bind to the receptor molecules 8 on the surface of the microspheres 6 and produce optical signals 12 that are measured. In Step 3, the photochromic composition in the microspheres are switched into colored forms by actinic radiation. In Step 4, a bright field illumination is used to capture the colored microspheres image and obtain the optical signature information of the microspheres.

The invention can be better appreciated by reference to the following specific examples.

### EXAMPLE 1

This example illustrates an array of micro-spheres containing a photo-chromic dye.

6-Nitrobenzospiropyran (0.1 grams) was dissolved in a mixture of 0.2 grams of toluene and 2.0 grams of acetone. 2.5 grams of an aqueous suspension of carboxylated polystyrene micro-spheres (4.2% solids and 9.5 μm diameter from Interfacial Dynamics Corporation, Portland, Oregon) was combined with 3.0 grams of acetone.

The above suspension of polystyrene micro-spheres in water and acetone was added to the solution of 6-nitrobenzospiropyram in acetone and toluene. The mixture was then filtered using a porous cotton filter (200 μm mesh size) and the filtrate was poured into a dialysis membrane tube (from Spectra/Por, 12,000-14000 molecular weight cutoff). The sample was washed with distilled water for one hour. After washing, the sample was once again filtered using a similar porous cotton filter, and the filtrate of dyed beads (containing 6-nitrobenzospiropyram) was collected.

50 μL of aqueous gelatin solution (4% w/w gelatin in water) were spread on a metallized (evaporated aluminum) polyethylene terephthalate (PET) support maintained at 40° C. using a coating knife (0.1 mm gap). The coating was chill set and dried at 5° C. 0.2 grams of the suspension of dyed beads prepared as above was diluted with 4 mL of water. 25 μL micro-liters of this diluted suspension were spread on top of the gelatin layer at 12° C. using a second coating knife (0.4 mm gap) to create a random array of dyed micro-spheres.

When the aforementioned array was illuminated with ultraviolet (UV) radiation having a wavelength of 366 nm using a Mineralight model UV6L-25 lamp from UVP Inc. San Gabriel, Calif.; the micro-spheres changed from a yellow color to blue. Furthermore, the micro-spheres retained their blue color even after the UV radiation had been turned off.

### EXAMPLE 2

This example illustrates a array of micro-spheres containing a photo-chromic compound in combination with a non-photochromic compound.

6-Nitrobenzospiropyram (0.1 grams) and 0.2 grams of dye Y1 were dissolved in a mixture of 0.2 grams of toluene and 2.0 grams of acetone. 2.5 grams of an aqueous suspension of carboxylated polystyrene micro-spheres (4.2% solids and 9.5 μm diameter from Interfacial Dynamics Corporation, Portland, Oreg.) was combined with 5.0 grams of acetone. The remainder of the procedure for preparing dye-loaded micro-spheres and coating these micro-spheres to create an array was similar to Example 1 except this time the array contained a mixture of dyed beads and non-dyed (clear) beads.

Color type and color levels in microspheres were analyzed using a hybrid analytical system comprising three parts: optical microscope, fluorescence microscope, and ultra-violet visible (UV-VIS) micro-spectrometer. This system uses high-intensity light, lenses, mirrors, apertures and optical detectors to first generate a magnified image of the coated array of microspheres. Such an image not only identifies the location of all microspheres 6 in FIG. 1, but it also enlarges the size of the bead to allow for the subsequent color detection process. The beads can be on transparent, black or mirrored support. Most preferably, the beads are coated on mirrored support.

Image of the bead array showing the location of the analyte emission is first obtained in an optical microscope set up (Olympus BX-30MFSP modular optical system, from Olympus PID Corp, Woodbury, N.Y., equipped with a Spot RT-Slider Camera, from Diagnostic Instruments, Inc.), operating under its fluorescence imaging mode, using fluorescence cubes, each consisting of an exciter filter, a dichroic mirror, and a barrier filter. This image, Image A, identifies the location of the analytes bound to receptor molecules on the microsphere, in a given field of view. Then, microspheres containing photochromic compounds, or a mixture of photochromic and non-photochromic compounds at different loading levels are exposed to actinic radiation to activate the photochromic dyes and generate color signatures. Then a second image, Image B, is then obtained in the same field of view to identify the location and color signature, of all the microspheres. Optical microscopy and fluorescence microscopy methods are broadly described by D. B. Murphy, “Fundamentals of Light Microscopy and Electronic Imaging”, Wiley-Liss, Inc. Publishing, 2001; and D. J. Goldstein, “Understanding the Light Microscope. A Computer-aided Introduction”, Academic Press, California, 1999.

Once the locations of all the microspheres are known, each bead (or only the bead tagged with the analyte) can then be analyzed by micro-spectroscopy to extract its color code. The spectral sensing apparatus is comprised of an F-40 light gathering optics setup (Filmetrics Inc., San Diego) that holds a 45° angled mirror etched with a small aperture. This feature permits the select extraction of spectral information from a specific region within a field of view in the optical microscope. The spectral information is then collected by the spectrometer sensor (USB-2000, OceanOptics, FL), and processed with the OOIBase32 software (OceanOptics, FL). Two-dimensional translation of the substrate, containing the microarray, allows a bead of interest to be positioned within the spectrometer aperture, based on its location as provided by Image A and Image B. Changes in the magnification of the microscope allows different amounts of the bead area to be confined by the aperture opening. For analysis of colors in these microspheres, it is preferred that at least two times the area defined by the diameter, D, of the bead is within the aperture opening, i.e., an area with squared length dimension, π(D/2)², containing the bead of interest. More preferably, one time the area based on the diameter of microsphere is used; and most preferably, 0.5 times the diameter region in the central portion of the bead is selected by the aperture opening.

To obtain the color type and color level of colorant in the microsphere, the spectral intensity response in the 300-1000 nm wavelength region of the electromagnetic radiation spectrum is collected, and processed, for example as absorbence, A, following the relationship:
A=log((I_{reference}−I_{background})/(Iₛₐₘₚₗₑ−I_{background}))
where
I_{reference}=intensity response of a bead without colorant,
I_{background}=null intensity with zero incident light
Iₛₐₘₚₗₑ=intensity response of colored bead
   It is known in the field that the absorbence, A, is related to the concentration of the light absorbing specie in the microsphere by Beer's Law:

A=ε b c, where ε is the molar absorptivity of the colorant in the bead,
b is the path length of the microsphere traversed by the light
c is the concentration of the colorant in the bead.
   Therefore,
   log((I_{reference}−I_{background})/(Iₛₐₘₚₗₑ−I_{background}))=ε b c

Hence, the measured intensity ratio is a monitor of the colorant concentration. The theory and practice of UV-VIS spectroscopy as followed in this disclosure is broadly described by D. A. Skoog and J. J. Leary, in the book

1. A color coded bead for use in a microarray for detecting target analytes, the bead comprising a photochromic compound in a medium, the bead having a receptor molecule on its surface;
wherein the photochromic compound confers on the bead a distinct optical signature; and
wherein the receptor molecule is capable of binding to a target analyte.

2. The bead as defined in claim 1 wherein the distinct optical signature is developed by actinic radiation.

3. The bead as defined in claim 1, wherein receptor molecule is biological or chemical.

4. The bead as defined in claim 1, wherein the bead comprises a mixture of different photochromic compounds.

5. The bead as defined in claim 1, wherein the bead comprises a mixture of photochromic and non-photochromic compounds.

6. The bead as defined in claim 1, wherein the distinct optical signature is produced by controlling a ratio of at least two photochromic compounds or a ratio of at least one photochromic compound and a non-photochromic compound.

7. The bead as defined in claim 1 wherein the distinct optical signature relates to the receptor molecule on its surface.

8. The bead of claim 1 wherein the medium is organic or inorganic.

9. The bead of claim 1 wherein the medium is polymeric.

10. The bead of claim 1 wherein the medium is an amorphous polymer.

11. The bead of claim 1 wherein the medium is polystyrene or poly(methylmethacrylate).

12. The bead as defined in claim 7 wherein the optical signal is fluorescence, absorbency, or chemiluminescence.

13. The bead as defined in claim 1 having a mean diameter of 1 to 50 microns.

14. The bead as defined in claim 1 having a mean diameter of 5 to 20 microns.

15. A microarray for detecting analytes, the microarray comprising a support, on which are disposed the beads defined in claim 1.

16. The microarray as defined in claim 15, wherein the beads are arranged on the support in random or in orderly distribution.

17. The microarray as defined in claim 15, wherein the beads are attached to the support by physical or chemical means.

18. The microarray of claim 15 wherein the support is modified to allow attachment of the microspheres.

19. The microarray of claim 13 wherein the support comprises polymer or glass.

20. The microarray of claim 13 wherein the laydown of microspheres on the support is 100 to a million per cm².

21. The microarray of claim 15 wherein the laydown of microspheres on the support is 10,000 to 100,000 per cm².

22. A method of identifying target analytes, the method comprising the steps of:
a) providing the microarray of claim 15, wherein the beads carry receptor molecules to which the target analytes can bind;
b) enabling the target analytes to bind to the receptor molecules thereby producing an optical signal;
c) detecting the optical signal, indicating presence of the targeted analytes on the bead;
d) subjecting the beads to actinic radiation;
e) interpreting the color change in the bead to identify the receptor molecule involved.

23. The method of claim 22 wherein the target analytes are labeled with optical emission tags.

24. A method of identifying target analytes, the method comprising the steps of:
a) providing the microarray of claim 15, wherein the beads carry receptor molecules capable of binding to the target analytes;
b) enabling the target analytes to bind to the receptor molecules thereby generating optical signals;
c) recording the signals as Image A;
d) activating the photochromic compounds in the beads into color signatures and recording them as Image B; and
e) matching Images A and B to determine the identity of the analyte.

25. The color coded bead of claim 1 wherein the photochromic compound is a dihydropyrene, a 1,4-2H-oxazine, a spirothiopyran, a naphthopyran, a triphenylmethane, a benzopyran, an azobenzene, a dithizone metal complex, a thioindigo, a spirooxazine, a spiropyran, a diarylethylene compound or a fulgide.

26. The color coded bead of claim 1 further including a light stabilizer.

27. The color coded bead of claim 1 wherein the light stabilizer is a plasticizer, a hindered amine, a hindered phenol or an excited state quencher.
